(19) United States
(12) Patent Application Publication
(10) Pub. No.: US 2004/0235922 A1
(43) Pub. Date: Nov. 25, 2004

(54) Compositions and methods for inducing adipose tissue cell death

(76) Inventors: Clifton A. Baile, Athens, GA (US); Mary Anne Della-Fera, La Veta, CO (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/845,912

(22) Filed: May 14, 2004

Related U.S. Application Data
(60) Provisional application No. 60/470,924, filed on May 15, 2003.

(51) Int. Cl. A61K 31/4172

(52) U.S. Cl. 514/396

(57) ABSTRACT

Pharmaceutical compositions, methods for increasing the rate of apoptosis in adipose tissue cells, and methods of reducing adipose tissue mass in a host, are described. One exemplary pharmaceutical composition, among others, includes at least one catecholamine in combination with a pharmaceutically acceptable carrier. The catecholamine is present in a dosage level effective to increase the rate of apoptosis in adipose tissue cells in a host.

### CROSS-REFERENCE TO RELATED APPLICATION

This application claims priority to co-pending U.S. provisional application entitled, “COMPOSITIONS AND METHODS FOR INDUCING ADIPOSE TISSUE CELL DEATH,” having Ser. No. 60/470,924, filed May 15, 2003, which is entirely incorporated herein by reference.

### TECHNICAL FIELD

The present disclosure is generally related to compositions and methods for administration of the compositions to hosts and, more particularly, is related to a compositions designed for treatment for inducing adipose tissue cell death and methods of administration thereof

### BACKGROUND

Obesity represents a major public health issue that continues to grow and accounts for 5.7% of total direct health care costs in the United States, and increases the risk of many of the leading causes of death (e.g., cardiovascular disease, diabetes, and cancer). Obesity is marked by excess adipose tissue (i.e., fat) accumulation arising from both an increased number of adipocytes and an increased size of adipocytes due to higher levels of lipid storage. Excess adipose tissue is strongly correlated with numerous health problems, including diabetes (e.g., decreased insulin sensitivity), vascular disease (e.g., hypertension), and certain forms of cancer. Exacerbating the health risks associated with obesity is that the most popular treatment for morbid obesity, liposuction, is a largely unregulated, half-billion dollar industry that exposes patients to additional health risks, including infection and death. As the current strategy for adipose reduction, liposuction is an invasive and painful procedure requiring costly equipment and considerable recovery time and often results in inconsistent tissue shape.

### SUMMARY

Briefly described, embodiments of this disclosure include pharmaceutical compositions, methods for increasing the rate of apoptosis in adipose tissue cells, and methods of reducing adipose tissue mass in a host. One exemplary pharmaceutical composition, among others, includes at least one catecholamine in combination with a pharmaceutically acceptable carrier. The catecholamine is present in a dosage level effective to increase the rate of apoptosis in adipose tissue cells in a host.

Another exemplary pharmaceutical composition, among others, includes at least one catecholamine in combination with a pharmaceutically acceptable carrier. The at least one catecholamine is present in a dosage level effective to reduce adipose tissue mass in a host.

One exemplary method for increasing the rate of apoptosis in adipose tissue cells, among others, includes administering an effective amount of at least one catecholamine to the host.

One exemplary method for reducing adipose tissue mass in a host, among others, includes administering to the host an effective amount of at least one catecholamine.

Other compositions, methods, features, and advantages of the present disclosure will be or become apparent to one with skill in the art upon examination of the following drawings and detailed description. It is intended that all such additional compositions, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a bar graph of retroperitoneal fat pad (Rp) weight, epididymal/parametrial fat pad (Epi/Par) weight and intrascapular brown adipose tissue (BAT) weight as a function of an administered does of a control, 200 ppm of clenbuterol, 200 ppm of ractopamine, and 800 ppm of ractopamine to treated mice. Bars having different letters indicate that the means are significantly different, p<0.05.

FIG. 2 illustrates a bar graph of percent DNA fragmentation (apoptosis) in epididymal/parametrial fat pads as a function of an administered dose of a control, 200 ppm of clenbuterol, 200 ppm of ractopamine, and 800 ppm of ractopamine to treated mice. Bars having different letters indicate that the means are significantly different, p<0.05.

FIG. 3 illustrates two bar graphs, where A) is daily weight gain (g) and B) is feed efficiency in male and female mice as a function of mice being fed 0 or 200 ppm of clenbuterol for 21 days. Bars having different letters indicate that the means are significantly different; a,b,c indicates a difference at p<0.05, while x, y, z indicates a difference at p<0.01.

FIG. 4 illustrates six bar graphs illustrating the effects of gender, genotype and treatment on weight (mg/g body wgt) of individual fat pads, skeletal muscles and heart. (RP, retroperitoneal fat pad; EPI/PAR, epididymal/parametrial fat pad; ING, inguinal fat pad; BAT, intrascapular brown adipose tissue; BF, biceps femoris; ST, semitendinosus; GC, gastrocnemius; TB, triceps brachii; HT, heart.) (*means are significantly different at p<0.05, **means are significantly different at p<0.01)

FIG. 5 illustrates a bar graph of EPI/PAR apoptosis (% DNA fragmentation) in female and male WT and GDF-8 KO mice as a function of mice fed 0 or 200 ppm of clenbuterol for 21 days. Bars having different letters indicate that the means are significantly different, p<0.05.

### DETAILED DESCRIPTION

Prior to describing the various embodiments of the present disclosure, the following provides definitions to various terms used within the disclosure.

The term “organism” or “host” refers to any living entity comprised of at least one cell. A living organism can be as simple as, for example, a single eukaryotic cell or as complex as a mammal, including a human being. As used herein, the term “host” includes humans, mammals (e.g., cats, dogs, horses, chicken, pigs, hogs, cows, and other cattle), and other living species that are in need of treatment. In particular, the term “host” includes humans, pigs, companion animals, and cattle.

The term “beta-adrenergic receptor (AR) agonist” means a compound, pharmaceutically acceptable salt, prodrug, or derivative thereof that demonstrates the ability to increase the rate of apoptosis in adipose tissue cells and/or reduce the mass of the adipose tissue.

The term “derivative” means a modification to the disclosed compounds.

The term “therapeutically effective amount” as used herein refers to that amount of the compound being administered, which will relieve to some extent one or more of the symptoms of the disorder being treated. In reference to adipose tissue, a therapeutically effective amount refers to that amount that has the effect of (1) causing apoptosis of adipose tissue and/or (2) reduce the mass of the adipose tissue.

“Pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

The term “pharmaceutically acceptable esters” as used herein refers to those esters of one or more compounds of the composition that are, within the scope of sound medical judgment, suitable for use in contact with the tissues of hosts without undue toxicity, irritation, allergic response, and the like, are commensurate with a reasonable benefit/risk ratio, and are effective for their intended use.

A “pharmaceutical composition” refers to a mixture of one or more of the compounds described herein, derivative, or pharmaceutically acceptable salts thereof, with other chemical components, such as physiologically acceptable carriers and excipients. One purpose of a pharmaceutical composition is to facilitate administration of a compound to the organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples of excipients include, without limitation, calcium carbonate, calcium phosphate, various sugars and types of starch, cellulose derivatives, gelatin, vegetable oils, and polyethylene glycols.

“Treating” or “treatment” of a condition includes preventing the condition from occurring in an animal that may be predisposed to the disease but does not yet experience or exhibit symptoms of the condition (prophylactic treatment), inhibiting the condition (slowing or arresting its development), providing relief from the symptoms or side-effects of the condition (including palliative treatment), and relieving the condition (causing regression of the condition).

The term “prodrug” refers to an agent that is converted into a biologically active form in vitro. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. 1. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharniacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):3′-48; D. M. Lambert (2000) Rationale and applications of lipids as prodrug carriers, Eur. J. Pharm. Sci., 11 Suppl 2: S15-27; Wang, W. et al. (1999) Prodrug approaches to the improved delivery of peptide drugs. Curr. Pharm. Des., 5(4):265-87.

As used herein, the term “topically active agents” refers to compositions of the present disclosure that elicit pharmacological responses at the site of application (contact) to a host.

As used herein, the term “topically” refers to application of the compositions of the present disclosure to the surface of the skin and mucosal cells and tissues.

The disclosed compounds can form salts that are also within the scope of this disclosure. Reference to each compound herein is understood to include reference to salts thereof, unless otherwise indicated. The term “salt(s),” as employed herein, denotes acidic and/or basic salts formed with inorganic and/or organic acids and bases. In addition, when a compound contains both a basic moiety and an acidic moiety, zwitterions (“inner salts”) may be formed and are included within the term “salt(s)” as used herein. Pharmaceutically acceptable (i.e., non-toxic, physiologically acceptable) salts are preferred, although other salts are also useful (e.g., in isolation or purification steps which may be employed during preparation). Salts of the compounds may be formed, for example, by reacting the compound with an amount of acid or base, such as an equivalent amount, in a medium such as one in which the salt precipitates or in an aqueous medium followed by lyophilization.

The disclosed compounds that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The disclosed compounds that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dihydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds are also contemplated herein. Solvates of the compounds are preferably hydrates.

To the extent that the disclosed compounds, and salts thereof, may exist in their tautomeric form, all such tautomeric forms are contemplated herein as part of the present disclosure.

All stereoisomers of the present compounds, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The chiral centers of the compounds of the present disclosure can have the S or R configuration as defined by the IUPAC 1974 Recommendations.

Description:

Embodiments of the present disclosure provide for compositions including at least one catecholamine, methods for inducing adipose tissue cell death, methods for reducing the mass of adipose tissue, and methods for treating obesity. In particular, the composition includes, but is not limited to, at least one catecholamine compound. In addition, embodiments of the methods include inducing adipose tissue cell death in a host by administering the composition. Further, embodiments of the methods reduce the mass of adipose tissue in the host by administering the composition. Also, the methods include treating conditions such as, but not limited to, obesity, in the host with the composition.

Apoptosis refers to a physiological process wherein selected cells are deleted in a rapid, efficient fashion through a signal-induced activation of endogenous self-destructive cellular processes. Apoptosis involves a sequence of distinct biochemical and morphological events characterized by DNA fragmentation, cell volume shrinkage, and production of plasma membrane-bounded apoptosis bodies, ultimately leading to cell death as described in more detail in Diabetes, Obesity and Metabolism, 3, 2001, 299-310.

In general, compositions of this disclosure can be used to induce and increase the rate of apoptosis of adipose tissue and/or reduce the mass of adipose tissue. In addition, compositions of this disclosure can be used to treat conditions such as, but not limited to, obesity and related conditions and diseases.

Catecholamine compounds (e.g., clenbuterol, a beta-adrenergic receptor (AR) agonist) have demonstrated the ability to increase the rate of apoptosis in adipose tissue cells, specifically white adipose tissue cells, in rat models. Clenbuterol, a beta 2-AR agonist, and ractopamine, a beta-1/beta-2-AR agonist, have been tested in male and female mice to determine their effects on growth, adiposity, and adipose tissue apoptosis. Diets containing control, 200 parts per million (ppm) clenbuterol, or 200 or 800 ppm ractopamine were fed for 21 days. Food intake (FI) was measured daily; body weight (BW) and temperatures (BT) were measured on days 0, 3, 7, 10, 14, 17, and 20. On day 21 mice were sacrificed, body composition was determined using PIXImus densitometry, and muscle and adipose tissues were collected. Neither clenbuterol nor ractopamine affected BT, FI, BW, feed efficiency or body composition. However, retroperitoneal (Rp) and epididymal/parametrial (Epi/Par) fat pad masses were reduced in both 800 ppm ractopamine (40±3 and 207±20 mg, respectively) and clenbuterol (35±7 and 211±22 mg) treated mice compared to control (66±8 and 319±30 mg, p<05). Brown adipose tissue (BAT) mass was greater (p<0.05) in clenbuterol treated mice compared to other treatments. Adipose tissue apoptosis (% DNA fragmentation) was increased in Epi/Par fat pads in clenbuterol (5.2±1.1%) and 800 ppm ractopamine (4.1±0.8%) treated mice compared to control (1.7±0.4%, p<0.05).

In a second study, the effect of clenbuterol on adiposity of male and female GDF-8 knockout (GDF-8 KO) (n=20) and wild type (WT) mice (n=20) was determined by feeding 0 or 200 ppm clenbuterol for 21 days. Clenbuterol increased weight gain (p<0.05), reduced epididymal/parametrial (EPI/PAR) fat pad weights (p<0.005) and increased adipocyte apoptosis in EPI/PAR and retroperitoneal (RP) fat pads (p<0.001). GDF-8 KO and WT mice responded similarly to clenbuterol.

These findings show that white adipose tissue (WAT) apoptosis can be induced by activation of beta-AR in both GDF-8 knockout and normal mice. In addition, these findings illustrate that beta-AR agonists may be used to control obesity. Additional details regarding the apoptosis of adipose tissue cells with catecholamine is described in more detail in Examples 1 and 2 below.

Designation of a catecholamine compound as interacting with or binding to a specific type of beta-adrenergic receptor varies from species to species. Therefore, the following designations are not intended to limit the compounds to a specific type of beta-adrenergic receptor, but rather to be used as a guide. Catecholamines are a general class of ortho-dihydroxyphenylalkylamines derived from tyrosine. Catecholamine compounds can include, but are not limited to, compounds that bind to beta-1-adrenergic receptors, beta-2-adrenergic receptors, both beta-1 and beta-2-adrenergic receptors, beta-3-adrenergic receptors, and combinations thereof. Compounds that bind to beta-1-adrenergic receptors include compounds such as, but not limited to, dobutamine and CGP20712A (benzamide, 2-hydroxy-5-[2-[[2-hydroxy-3-[4-[1-methyl-4-(trifluoromethyl)-1H-imidazol-2-yl]phenoxy]propyl]amino]ethoxy]-, monomethanesulfonate (salt) (9CI)). Compounds that bind to beta-2-adrenergic receptors include compounds such as, but not limited to, clenbuterol, cimaterol, ICI 118551 (2-butanol, 1-[(2,3-dihydro-7-methyl-1H-inden-4-yl)oxy]-3-[(1-methylethyl)amino]-, (2R,3S)-rel-(9CI)), procaterol, terbutaline, formoterol, salmeterol, and salbutamol. Compounds that bind to beta-1/beta-2-adrenergic receptors include compounds such as, but not limited to, ractopamine. Compounds that bind to beta-3-adrenergic receptors include compounds such as, but not limited to, CL-316243 (1,3-benzodioxole-2,2-dicarboxylic acid, 5-[(2R)-2-[[(2R)-2-(3-chlorophenyl)-2-hydroxyethyl]amino]propyl]-, disodium salt (9CI)), BRL 37344 (acetic acid, [4-[(2R)-2-[[(2R)-2-(3-chlorophenyl)-2-hydroxyethyl]amino]propyl]phenoxy]-, rel-(9CI)), CGP 12177 (2H-benzimidazol-2-one, 4-[3-[(1,1-dimethylethyl)amino]-2-hydroxypropoxy]-1,3-dihydro-(9CI)), and BRL 26830A (benzoic acid, 4-[(2R)-2-[[(2R)-2-hydroxy-2-phenylethyl]amino]propyl]-, methyl ester, rel-, (2E)-2-butenedioate (2:1) (salt) (9CI)).

Where such forms exist, catecholamine compounds may include catecholamine analogues, homologues, isomers, or derivatives thereof, that function to induce adipose tissue cell death and/or reduce adipose tissue mass in a host. In addition, catecholamine compounds can include pharmaceutically acceptable salts, esters, and prodrugs of the catecholamine compounds described above.

Embodiments of the present disclosure include methods for inducing apoptosis of adipose tissue and/or reduce the mass of adipose tissue in a host. In addition, embodiments of this disclosure include methods to treat conditions such as, but not limited to, obesity and related conditions and diseases in a host.

Pharmaceutical compositions and dosage forms of the disclosure include a pharmaceutically acceptable salt of the compound and/or a pharmaceutically acceptable polymorph, solvate, hydrate, dehydrate, co-crystal, anhydrous, or amorphous form thereof. Specific salts of disclosed compounds include, but are not limited to, sodium, lithium, potassium salts, and hydrates thereof.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a dosage form used in the acute treatment of a condition or disorder may contain larger amounts of the active ingredient, e.g., the disclosed compounds or combinations thereof, than a dosage form used in the chronic treatment of the same condition or disorder. Similarly, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical pharmaceutical compositions and dosage forms include one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that include primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. A specific solubility modulator is tartaric acid.

Like the amounts and types of excipients, the amounts and specific type of active ingredient in a dosage form may differ depending on factors. It will be understood, however, that the total daily usage of the compositions of the present disclosure will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed, the age, body weight, general health, sex, and diet of the host; the time of administration; route of administration; rate of excretion of the specific compound employed; the duration of the treatment; drugs used in combination or coincidental with the specific composition employed; and like factors well known in the medical arts. For example, it is well within the skill of the art to start doses of the composition at levels lower than those required to achieve the desired therapeutic effect and to gradually increase the dosage until the desired effect is achieved.

Compositions of the present disclosure are preferably formulated in dosage unit form for ease of administration and uniformity of dosage. “Dosage unit form” as used herein refers to a physically discrete unit of the composition appropriate for the host to be treated. Each dosage should contain the quantity of composition calculated to produce the desired therapeutic affect either as such, or in association with the selected pharmaceutical carrier medium.

Preferred unit dosage formulations are those containing a daily dose or unit, daily sub-dose, or an appropriate fraction thereof, of the administered ingredient. For example, approximately 3 to 30 milligrams/kilogram per day of a catecholamine compound (e.g., clenbuterol) can reduce the mass of the adipose tissue in a mouse. In particular, administration of approximately 28 milligrams/kilogram daily of the catecholamine compound reduces the mass of the adipose tissue about 34 percent, as discussed in more detail in Examples 1 and 2. These results can be used to predict an approximate amount of the catecholamine compound to be administered to a human or cattle.

The approximation includes host factors such as surface area, weight, metabolism, tissue distribution, absorption rate, and excretion rate, for example. Therefore, approximately 1 to 10 milligrams/kilogram per day of the catecholamine compound should produce similar results in humans. In particular, approximately 3 milligrams/kilogram per day of the catecholamine compound can be administered to humans to produce similar results. As stated above, a therapeutically effective dose level will depend on many factors, as described above. In addition, it is well within the skill of the art to start doses of the composition at relatively low levels, and increase the dosage until the desired effect is achieved.

In addition, approximately 0.02 to 0.2 milligrams/kilogram per day of the catecholamine compound should produce similar results in cattle. In particular, approximately 0.02 milligrams/kilogram per day of the catecholamine compound can be administered to cattle. As stated above, a therapeutically effective dose level will depend on many factors, as described above. In addition, it is well within the skill of the art to start doses of the composition at relatively low levels, and increase the dosage until the desired effect is achieved.

In addition, approximately 0.1 to 2 milligrams/kilogram per day of the catecholamine compound should produce similar results in dogs. In particular, approximately 1 milligram/kilogram per day of the catecholamine compound can be administered to dogs. As stated above, a therapeutically effective dose level will depend on many factors, as described above. In addition, it is well within the skill of the art to start doses of the composition at relatively low levels, and increase the dosage until the desired effect is achieved.

In addition, approximately 0.03 to 1 milligrams/kilogram per day of the catecholamine compound should produce similar results in cats. In particular, approximately 0.1 milligrams/kilogram per day of the catecholamine compound can be administered to cats. As stated above, a therapeutically effective dose level will depend on many factors, as described above. In addition, it is well within the skill of the art to start doses of the composition at relatively low levels, and increase the dosage until the desired effect is achieved.

In addition, approximately 1 to 10 milligrams/kilogram per day of the catecholamine compound should produce similar results in chickens. In particular, approximately 1 milligram/kilogram per day of the catecholamine compound can be administered to chickens. As stated above, a therapeutically effective dose level will depend on many factors, as described above. In addition, it is well within the skill of the art to start doses of the composition at relatively low levels, and increase the dosage until the desired effect is achieved.

In addition, approximately 0.01 to 1 milligrams/kilogram per day of the catecholamine compound should produce similar results in pigs. In particular, approximately 0.01 milligrams/kilogram per day of the catecholamine compound can be administered to pigs. As stated above, a therapeutically effective dose level will depend on many factors, as described above. In addition, it is well within the skill of the art to start doses of the composition at relatively low levels, and increase the dosage until the desired effect is achieved.

Pharmaceutical compositions of the disclosure that are suitable for oral administration can be presented as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 118th ed., Mack Publishing, Easton, Pa. (1990).

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms, depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies, based upon the type of formulation and mode of administration, and is readily discernible to those of ordinary skill in the art. Typical pharmaceutical compositions comprise from about 0.5 to 15 weight percent of disintegrant, or from about 1 to 5 weight percent of disintegrant.

Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients that are well known in the art and are listed in the USP (XXI)/NF (XVI), which is incorporated herein by reference. In general, lactose-free compositions comprise a pharmaceutically acceptable salt of an catecholamine compound, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Pharmaceutically acceptable salts of the disclosed compounds can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. See, e.g., Kim, Cherng-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm & Haas, Spring House, Pa. USA).

One embodiment of the disclosure encompasses a unit dosage form which comprises a pharmaceutically acceptable salt of the disclosed compounds (e.g., a sodium, potassium, or lithium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. No. 6,375,978 B1; No. 6,368,626 B1; No. 6,342,249 B1; No. 6,333,050 B2; No. 6,287,295 B1; No. 6,283,953 B1; No. 6,270,787 B1; No. 6,245,357 B1; and No. 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™, and Push-Stick™ Systems, all of which are well known. See, e.g. worldwide website alza.com. Additional OROS® systems that can be used for the controlled oral delivery of compounds and compositions of the disclosure include OROS®-CT and L-OROS®; see, Delivery Times, vol. 11, issue II (Alza Corporation).

Conventional OROS® oral dosage forms are made by compressing a drug powder into a hard tablet, coating the tablet with cellulose derivatives to form a semi-permeable membrane, and then drilling an orifice in the coating (e.g., with a laser). Kim, Cherng-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively delivery drugs with low water solubility. This disclosure doses, encompass the incorporation of the compounds and salts thereof, non-salt isomers and isomeric mixtures thereof and the like into OROS® dosage forms.

A specific dosage form of the compositions of the disclosure includes at least the following: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes the compound, a salt thereof, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the disclosure includes at least the following: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation includes the compound, a salt thereof, or a polymorph, solvate, hydrate, dehydrate, co-crystal, anhydrous, or amorphous form thereof (See U.S. Pat. No. 6,342,249, the entirety of which is incorporated herein by reference.)

Parenteral dosage forms can be administered to patients by various routes, including, but not limited to, subcutaneous, intravenous (including bolus injection), intramuscular, and intraarterial. Since administration of parenteral dosage forms typically bypasses the host's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; Water for Injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, benzyl benzoate, and mixtures thereof.

Topical dosage forms of the disclosure include, but are not limited to, creams, lotions, ointments, gels, shampoos, sprays, aerosols, solutions, emulsions, and other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990).

Transdermal and mucosal dosage forms of the compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of the compounds of the disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of the compounds can be used to further adjust the properties of the resulting composition.

Typically, active ingredients of the pharmaceutical compositions of the disclosure are preferably not administered to a patient at the same time or by the same route of administration. This disclosure therefore encompasses kits which, when used by the medical practitioner, can simplify the administration of appropriate amounts of active ingredients to a patient.

A typical kit includes a unit dosage form of a pharmaceutically acceptable salt of the compound. In particular, the pharmaceutically acceptable salt of the compound is the sodium, lithium, or potassium salt, or a polymorph, solvate, hydrate, dehydrate, co-crystal, anhydrous, or amorphous form thereof A kit may further include a device that can be used to administer the active ingredient. Examples of such devices include, but are not limited to, syringes, drip bags, patches, and inhalers.

Kits of the disclosure can further include pharmaceutically acceptable vehicles that can be used to administer one or more active ingredients. For example, if an active ingredient is provided in a solid form that must be reconstituted for parenteral administration, the kit can include a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Having summarized embodiments, reference will now be made in detail to two illustrative examples. While the disclosure is described in connection with these examples, there is no intent to limit the embodiments of the disclosure to the following examples. On the contrary, the intent is to cover all alternatives, modifications, and equivalents included within the spirit and scope of the disclosure. Example 1 is described in greater detail in (Page, K. A., D. L. Hartzell, C. Li, A. L. Westby, M. A. Della-Fera, M. J. Azain, T. D. Pringle, and C. A. Baile, Beta-Adrenergic Receptor Agonists Increase Apoptosis of Adipose Tissue In Mice. Domest Anim Endocrinol, 2004. 26: p. 23-31.), while Example 2 is described in greater detail in (Page, K., M. A. Della-Fera, C.-L. Li, M. J. Azain, T. D. Pringle, D. Hartzell, and C. A. Baile, Effects of Clenbuterol on Muscle, Adipose Tissue and Adipocyte Apoptosis in Myostatin Knockout Mice. J Anim Sci, 2004 in press.).

### EXAMPLE 1

The effects of clenbuterol, a β2-AR agonist, and ractopamine, a β1/β2-AR agonist, on growth, adiposity and adipose tissue apoptosis in male and female mice have been tested by feeding diets containing control, 200 ppm clenbuterol, or 200 or 800 ppm ractopamine. Food intake (FI) was measured daily; body weight (BW) and temperatures (BT) were measured on days 0, 3, 7, 10, 14, 17, and 20. On day 21 mice were sacrificed, body composition was determined using PIXImus densitometry, and muscle and adipose tissues were collected. There were no treatment effects on BT, FI, BW, feed efficiency or body composition. Retroperitoneal (Rp) and epididymal/parametrial (Epi/Par) fat pad masses were reduced in both 800 ppm ractopamine (40±3 and 207±20 mg, respectively) and clenbuterol (35±7 and 211±22 mg) treated mice compared to control (66±8 and 319±30 mg, p<0.05). Brown adipose tissue (BAT) mass was greater (p<0.05) in clenbuterol treated mice compared to other treatments. Adipose tissue apoptosis (% DNA fragmentation) was increased in Epi/Par fat pads in clenbuterol (5.2+1.1%) and 800 ppm ractopamine (4.1±0.8%) treated mice compared to control (1.7+0.4%, p<0.05). These findings show that WAT apoptosis can be induced by activation of βAR in mice, although the mechanism is unknown.

Materials and Methods

Animals: Five week old male (n=20) and female (n=20) ICR mice (Harlan Research Laboratories, Indianapolis, Ind.) were allowed to acclimate for one week prior to the experiment start date. Animals were housed singly in suspended wire cages and were provided ground rodent chow (ProLab® RMH 2500; Purina Mills, St. Louis, Mo.) and water ad libitum. Ambient room temperature was maintained and the light/dark cycle was 0600/1800 hours, respectively. Animals were cared for in accordance with the Guide for the Care and Use of Laboratory Animals (Institute of Laboratory Animal Resources Commission on Life Sciences National Research Council. Guide for the Care and Use of Laboratory Animals. National Academy of Sciences, 1996). Prior to the start of the study, animals were briefly anesthetized with isofluorane and an IPPT-200 transponder (programmable ID and temperature, BMDS, Seaford, Del.) was implanted subcutaneously between the scapulae.

Materials: Test articles included clenbuterol (Sigma Chemical Company, St. Louis, Mo., item # C5423) and ractopamine hydrochloride—Paylean® 9 (Elanco Animal Health, Indianapolis, Ind.). Clenbuterol is available as a pure product, and ractopamine is readily available as a type A medicated article. Paylean® 9 contains about 20 g ractopamine hydrochloride per kilogram in a ground corncob base. In order to achieve doses of 200 and 800 ppm it was necessary to add about 20 and 80 g of product per kilogram of diet. Control and clenbuterol diets were prepared with about 80 g of alphacel, which is a diluent used to mimic the effect of the corncob in the ractopamine product. The dose of clenbuterol used in this experiment was similar to those used in previous experiments with mice (Agbenyega E T, and Wareham A C. Effect of clenbuterol on skeletal muscle atrophy in mice induced by the glucocorticoid dexamethasone. Comp Biochem Physiol Comp Physiol 1992;102:141-145, Rothwell N. J., and Stock M J. Modification of body composition by clenbuterol in normal and dystrophic (mdx) mice. Biosci Rep 1985;5:755-760, Hyltander A, Svaninger G, and Lundholm K. The effect of clenbuterol on body composition in spontaneously eating tumour-bearing mice. Biosci Rep 1993;13:325-331, Hayes A, and Williams DA. Long-term clenbuterol administration alters the isometric contractile properties of skeletal muscle from normal and dystrophin-deficient mdx mice. Clin Exp Pharmacol Physiol 1994;21:757-765, Dupont-Versteegden EE. Exercise and clenbuterol as strategies to decrease the progression of muscular dystrophy in mdx mice. J Appl Physiol 1996;80:734-741).

Daily Observations: Food intake was measured daily, while body weight and body temperatures were measured on days 0, 3, 7, 10, 14, 17, and 20 at approximately the same time of day (1300 hours) during the treatment period. At 9 weeks of age (day 21), a final body weight measurement was obtained approximately one hour prior to euthanasia, and animals were sacrificed by decapitation following CO₂ asphyxiation.

Body composition analysis was performed after the mice had been decapitated, using a PIXImus® densitometer (GE Lunar Corporation; Waukesha, Wis.), which uses dual-energy x-ray absorptiometry to measure whole body (subcranial) bone mineral density, bone mineral content, percent lean tissue and percent fat tissue. Brown intrascapular adipose tissue (BAT), inguinal (Ing), retroperitoneal (Rp), and epididymal (Epi) or parametrial (Par) white adipose tissues were harvested. Heart, liver, and kidney, as well as the right-side gastrocnemius (GC), semitendinosus, biceps femoris, triceps brachii, and longissimus dorsi muscles were removed, weighed individually, flash frozen in liquid nitrogen and then stored at −80° C. Tissue weights were recorded for statistical analysis.

DNA Isolation and Apoptosis Assay: Only the Epi and Par fat pads had sufficient tissue for the apoptosis assay. Apoptosis was assayed in two ways: DNA isolated from fat tissue was separated into two fractions: fragmented and genomic DNA. First, the fragmented DNA was run on an agarose gel in order to identify a ladder pattern of internucleosomal DNA degradation that is characteristic of apoptosis. Second, apoptosis was quantified as the ratio of fragmented- to total-DNA, multiplied by 100. Briefly, approximately 50 mg of the Epi or Par white adipose tissue was homogenized in lysis buffer (10 mM Tris-HCL, pH 8.0, 10 mM EDTA, pH 8.0; 0.5% Triton X-100) and centrifuged at 14,000× g for 15 min to separate fragmented DNA from genomic DNA. The supernatant, containing fragmented DNA, was extracted with phenol-chloroform-isoamyl alcohol (25:24:1), and the DNA was precipitated by adding polyacryl carrier (Molecular Research Center, Inc., Cincinnati, Ohio) and ethanol. Genomic (non-fragmented) DNA was extracted from the pellet with DNAzol and the polyacryl carrier. DNA in each fraction was quantified by the PicoGreen method (Molecular Probes, Inc., Eugene, Oreg.) and fluorescence was measured using a SpectroMax Gemini (Molecular Devices).

Statistical Analysis: Analysis of variance (SAS) was used to determine significance of treatment and gender effects. Significance among means was determined by Tukey's test. Statistically significant differences are defined at the 95% confidence interval.

Results

Food Intake, Body Weight, Body Composition and Body Temperature: There were no significant gender, treatment or gender x treatment effects on body weight, weight gain, food intake, feed efficiency or body temperature. Densitometry indicated no significant gender, treatment or gender x treatment differences in body composition (Table 1).

TABLE 1
Effects of 21 Day Oral Treatment With Control, 200 ppm Clenbuterol, 200 ppm
Ractopamine and 800 ppm Ractopamine on Body Weight, Food Intake, Feed
Efficiency, Body Temperature and Body Composition in Mice.

| | | Clenbuterol | Ractopamine | Ractopamine | |
|---|---|---|---|---|---|
| | Control | 200 ppm | 200 ppm | 800 ppm | T_{SE} |
| Initial Body Weight (g) | 23.4 | 22.3 | 23.4 | 23.2 | 0.6 |
| Weight Gain (g) | 7.44 | 8.90 | 8.25 | 8.32 | 0.79 |
| Final Body Weight (g) | 30.75 | 32.1 | 31.4 | 31.5 | 0.70 |
| Body Temp (° C.) | 37.2 | 36.2 | 37.0 | 36.8 | 0.60 |
| Cumulative Feed | 5.79 | 6.10 | 5.92 | 6.39 | 0.19 |
| Intake (g) | | | | | |
| Feed Efficiency | 16.4 | 14.4 | 15.8 | 16.6 | 1.17 |

Densitometry

| | | | | | |
|---|---|---|---|---|---|
| Lean (g) | 25.68 | 26.80 | 25.63 | 27.42 | 0.8 |
| Fat (g) | 3.10 | 2.81 | 3.00 | 3.01 | 0.20 |
| Bone Mineral | 0.67 | 0.65 | 0.67 | 0.67 | 0.004 |
| Content (g) | | | | | |

There were no significant differences among treatment means.
Data Shown Are Treatment Means.
T_{SE}: Treatment Standard Error

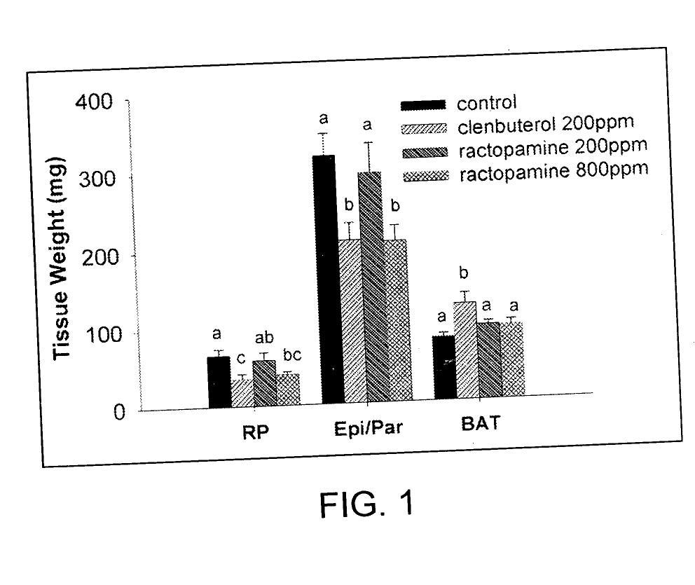

Tissue Weights: There were significant gender effects for weights of Rp adipose tissue, biceps brachialis muscle, heart, kidney and liver, and significant treatment effects for Rp and Epi/Par adipose tissue and BAT (Table 2 and FIG. 1). There was a significant gender x treatment interaction for the Epi/Par fat pad (p<0.05; Table 3). Overall, Rp and Epi/Par fat pad weights were reduced in both 800 ppm ractopamine and clenbuterol treated mice compared to control (p<0.05; FIG. 1), but for the Epi/Par fat pads, the effect occurred only in females (Table 3). BAT mass was significantly increased (p<0.05) in the clenbuterol-treated group compared to control (FIG. 1).

TABLE 2
Effects of 21 Day Oral Treatment With Control, 200 ppm Clenbuterol, 200 ppm
Ractopamine and 800 ppm Ractopamine on Selected Tissue Weights in Male and Female
Mice (N = 10).

| Gender | | Rp | Epi/Par | Inguinal | BAT | biceps | heart | kidney | liver |
|---|---|---|---|---|---|---|---|---|---|
| Males | Mean | .058 | .272 | .2 | .106 | .154 | .164 | .672 | 2.41 |
| | N | 20 | 20 | 20 | 20 | 18 | 20 | 20 | 20 |
| | SEM | .006 | .02 | .01 | .008 | .005 | .005 | .014 | .05 |
| Females | Mean | .041 | .245 | .180 | .092 | .133 | .145 | .523 | 1.98 |
| | N | 20 | 20 | 20 | 20 | 20 | 20 | 20 | 20 |
| | SEM | .005 | .922 | .016 | .005 | .008 | .004 | .014 | .061 |
| Gender | P | <.05 | <.05 | <.01 | NS | NS | <.01 | <.01 | <.01 |
| Treatment | | Rp | Epi/Par | Ing | BAT | Biceps | Heart | Kidney | Liver |
| Control | Mean | .065 | .319 | .207 | .081 | .141 | .151 | .595 | 2.20 |
| | SEM | .008 | .03 | .02 | .005 | .004 | .006 | .032 | .105 |
| Clenbuterol | Mean | .035 | .211 | .150 | .124 | .145 | .164 | .582 | 2.14 |
| | SEM | .007 | .021 | .022 | .014 | .009 | .008 | .026 | .072 |
| Rac200 | Mean | .058 | .295 | .230 | .096 | .128 | .147 | .586 | 2.16 |
| | SEM | .009 | .039 | .025 | .005 | .008 | .005 | .032 | .118 |
| Rac800 | Mean | .039 | .207 | .176 | .095 | .158 | .153 | .612 | 2.23 |
| | SEM | .003 | .019 | .020 | .007 | .015 | .008 | .034 | .033 |
| Treatment | P | <.05 | <.01 | NS | <.01 | NS | NS | NS | NS |

Data Shown Are Treatment Means.
SEM: Standard Error of the Mean

TABLE 3
Effects of 21 Day Oral Treatment With Control, 200 ppm
Clenbuterol, 200 ppm Ractopamine and 800 ppm Ractopamine
on Epi and Par Weights in Male and Female Mice (N = 5).

| Males | | | Females | | |
|---|---|---|---|---|---|
| Treatment | Mean | SEM | Treatment | Mean | SEM |
| Control | .27^{ab} | .031 | Control | .36^{a} | .045 |
| Clen | .24^{ab} | .022 | Clen | .18^{b} | .034 |
| Rac200 | .36^{a} | .062 | Rac200 | .22^{b} | .023 |
| Rac800 | .19^{b} | .018 | Rac800 | .22^{b} | .032 |

^{a,b}Means with different superscripts are different, p < .05
Adipose Tissue Apoptosis: There was no significant gender effect on % DNA

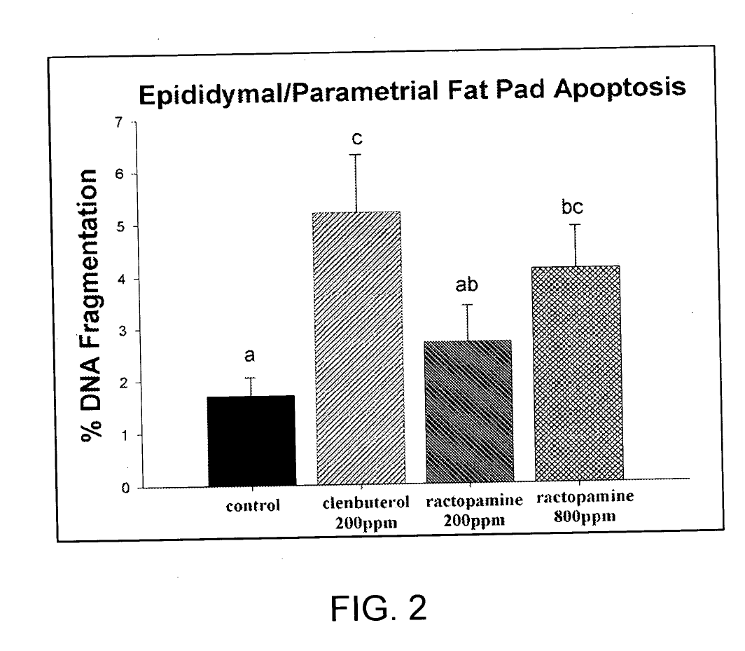

fragmentation in the Epi/Par fat pad, but there was a significant treatment effect (F(3,30)=4.0; p=0.017). Percent DNA fragmentation in the Epi/Par fat pad in the 200 ppm clenbuterol-treated group was increased compared to control and 200 ppm ractopamine-treated groups (p<0.05; FIG. 2). There was also a significant increase in % DNA fragmentation in the 800 ppm ractopamine treatment group compared to control (p<0.05; FIG. 2). Neither clenbuterol nor ractopamine treatments increased DNA fragmentation in retroperitoneal or inguinal fat pads.

Discussion:

Treatment of male and female mice with either 200 ppm clenbuterol or 800 ppm ractopamine in the diet resulted in a significant increase in apoptosis in the epididymal and parametrial adipose tissues, a finding that has not been previously reported. This finding suggests that activation of β-adrenergic receptors can trigger the apoptotic process in adipose tissue. Although the cell type involved was not specifically identified, these results are similar to those in previous experiments in which leptin administration was shown to increase apoptosis of adipocytes (Qian H, Azain M J, Compton M M, Hartzell D L, Hausman G J, and Baile C A. Brain administration of leptin causes deletion of adipocytes by apoptosis, Endocrinology 1998;139:791-794) and decrease the number of adipocytes in fat pads in which apoptosis was found (Gullicksen P S, Hausman D B, Dean R G, Hartzell D L, and Baile C A. Adipose tissue cellularity and apoptosis after intracerebroventricular injections of leptin and 21 days of recovery in rats, International Journal of Obesity 2002; in press). Clenbuterol has been shown to have direct effects on mouse adipocytes in previous studies. For example, clenbuterol stimulated lipolysis and inhibited insulin-induced lipolysis in mouse adipocytes in vitro (Orcutt A L, Cline T R, and Mills S E. Influence of the beta 2-adrenergic agonist clenbuterol on insulin-stimulated lipogenesis in mouse adipocytes, Domest Anim Endocrinol 1989;6:59-69), and clenbuterol decreased insulin binding to mouse adipocytes in vitro (Dubrovin L C, Liu C Y, and Mills S E. Insulin binding to mouse adipocytes exposed to clenbuterol and ractopamine in vitro and in vivo, Domest Anim Endocrinol 1990;7:103-109). These findings suggest that the effects of clenbuterol and ractopamine on adipocyte apoptosis may also be direct effects.

Clenbuterol treatment also resulted in increased BAT mass in both males and females and decreased mass of Rp and Epi/Par fat pads in females only, findings that are similar to those of a previous study with mice (Eisen E J, Croom W J, Jr., and Helton S W. Differential response to the beta-adrenergic agonist cimaterol in mice selected for rapid gain and unselected controls, J Anim Sci 1988;66:361-371). It was also found that 800 ppm ractopamine resulted in decreased Rp and Epi/Par fat pad mass. The increase in BAT mass in clenbuterol treated mice is consistent with the increase that occurs as a result of increased sympathetic nervous system (SNS) stimulation during cold exposure (Lindquist J M, and Rehnmark S. Ambient temperature regulation of apoptosis in brown adipose tissue. Erk1/2 promotes norepinephrine-dependent cell survival, J Biol Chem 1998; 273:30147-30156) and after exogenous administration of specific β-AR agonists (Nagase I, Sasaki N, Tsukazaki K, Yoshida T, Morimatsu M, and Saito M. Hyperplasia of brown adipose tissue after chronic stimulation of beta 3-adrenergic receptor in rats, Jpn J Vet Res 1994;42:137-145). Phosphorylation of the mitogen-activated protein kinase ERK1/2 has been shown to be involved in the hyperplastic effect of β-AR stimulation of BAT (J Biol Chem 1998; 273:30147-30156).

Body composition, food intake, feed efficiency, body weight, and body temperature were not significantly affected by either ractopamine or clenbuterol in this study. Likewise, neither β-AR agonist increased muscle mass.

The effect of clenbuterol and ractopamine on adipose apoptosis is interesting. Clenbuterol has been shown to protect against apoptosis in both brain and liver (Zhu Y, Culmsee C, Semkova I, and Krieglstein J. Stimulation of beta2-adrenoceptors inhibits apoptosis in rat brain after transient forebrain ischemia, J Cereb Blood Flow Metab 1998;18:1032-1039, Zhu Y, Prehn J H, Culmsee C, and Krieglstein J. The beta2-adrenoceptor agonist clenbuterol modulates Bcl-2, Bcl-xl and Bax protein expression following transient forebrain ischemia, Neuroscience 1999;90:1255-1263, and Andre C, Couton D, Gaston J, Erraji L, Renia L, Varlet P, Briand P, and Guillet J G. Beta2-adrenergic receptor-selective agonist clenbuterol prevents Fas-induced liver apoptosis and death in mice, Am J Physiol 1999;276:G647-654), and in BAT, β-AR stimulation also protects against adipocyte apoptosis (J Biol Chem 1998; 273:30147-30156). In contrast, norepinephrine, a β-AR agonist, was found to induce apoptosis of cardiac myocytes via β1-AR stimulation (Singh K, Xiao L, Remondino A, Sawyer D B, and Colucci W S. Adrenergic regulation of cardiac myocyte apoptosis, J Cell Physiol 2001;189:257-265, Singh K, Communal C, Sawyer D B, and Colucci W S. Adrenergic regulation of myocardial apoptosis, Cardiovasc Res 2000;45:713-719). However, the effect of β-AR agonists on apoptosis of white adipose tissue has not previously been reported.

### EXAMPLE 2

The effects of clenbuterol on muscularity and adiposity in male and female GDF-8 knockout (GDF-8 KO) (n=20) and wild type (WT) mice (n=20) were tested by feeding 0 or 200 ppm clenbuterol for 21 days. Analysis of main effects showed that clenbuterol treatment increased weight gain (p<0.05), reduced epididymal/parametrial (EPI/PAR) fat pad weights (p<0.005), increased heart weights (p<0.03), and increased adipocyte apoptosis in EPI/PAR and retroperitoneal (RP) fat pads (p<0.001). There was a trend for clenbuterol to increase feed efficiency (p=0.07), and a significant gender x treatment interaction showed that clenbuterol more than doubled feed efficiency in males (p<0.01), but had no effect in females. For all other variables, GDF-8 KO and WT mice responded similarly to clenbuterol. Comparison of genotypes showed that GDF-8 KO mice had greater bone mineral density, bone mineral content, and lean tissue than WT mice (p<0.04), as determined by PIXImus densitometry. Thus, the lack of GDF-8 does not alter sensitivity to β2-AR stimulation.

Materials and Methods

Animals: Twelve-week old male (n=10) and female (n=10) GDF-8 (−/−) knockout mice and male (n=10) and female (n=10) GDF-8 (+/+) WT mice were used in the study. GDF-8 knockout mice, which were derived from the 129/SvJ mouse strain, were originally obtained from MetaMorphix Inc. and were bred in-house for the study. Mice were housed singly in suspended wire cages in a room maintained at 22±1 C and with a 12:12 hour light/dark cycle. Mice were provided ground rodent chow (ProLab® RMH 2500; Purina Mills, St. Louis, Mo.) and water ad libitum. Mice were cared for in accordance with the Guide for the Care and Use of Laboratory Animals.

Materials: Clenbuterol (Sigma Chemical Company, St. Louis, Mo., item# C5423), was thoroughly mixed into the ground rodent chow to provide a 200 ppm concentration.

Daily Observations: Food intake was measured daily, and body weight was measured on days 0, 3, 7, 10, 14, 17, 20, and 21 at approximately 1430 h during the treatment period. At 15 weeks of age (day 21), a final body weight measurement was obtained approximately one hour prior to euthanasia, and mice were sacrificed by decapitation following CO₂ asphyxiation.

Body composition analysis was performed using a dual-energy x-ray PIXImus® densitometer (GE Lunar Corporation; Waukesha, Wis.). The data obtained included bone mineral density (BMD), bone mineral content (BMC), body surface area (area, cm²) lean weight (g), fat weight (g) and fat percent. The x-ray instrument has moderately low energy and a high resolution (80/35 kVp and 0.18×0.18 mm pixel size). The densitometer was calibrated using an aluminum and lucite phantom prior to scanning the mice. After scanning, brown intrascapular adipose tissue (BAT), inguinal (ING), retroperitoneal (RP), and epididymal (EPI) or parametrial (PAR) white adipose tissue, as well as heart (HT), liver, kidney, the right-side gastrocnemius (GC), semitendinosus (ST), biceps femoris (BF), and triceps brachii (TB) were harvested. After removal, each tissue was weighed individually; all tissues except adipose were flash frozen in liquid nitrogen and then stored at −80° C. (within 15 min of death). Tissue weights were recorded for statistical analysis.

DNA Isolation and Apoptosis Assay: DNA was isolated from fresh fat tissues and separated into two fractions: fragmented and genomic DNA. The samples were first run on an agarose gel to identify a ladder pattern of internucleosomal DNA degradation, which is characteristic of apoptosis (Qian, H., M. J. Azain, M. M. Compton, D. L. Hartzell, G. J. Hausman, and C. A. Baile, Brain administration of leptin causes deletion of adipocytes by apoptosis. Endocrinology, 1998. 139(2): p. 791-4). Apoptosis was then quantified as the ratio of fragmented- to total-DNA as previously described (Gullicksen, P. S., R. G. Dean, and C. A. Baile, Detection of DNA Fragmentation and Apoptotic Proteins, and Quantification of Uncoupling Protein Expression by Real-Time RT-PCR in Adipose Tissue. J Biochem Biophys Methods, 2004. 58(1): p. 1-13). Briefly, approximately 50 mg of EPI/PAR or RP adipose tissue from each animal was homogenized in lysis buffer (10 mM Tris-HCL, pH 8.0; 10 mM EDTA, pH 8.0; 0.5% Triton X-100) and centrifuged at 14,000×g for 15 minutes to separate fragmented DNA from genomic DNA. The supernatant, containing fragmented DNA, was extracted with phenol-chloroform-isoamyl alcohol (25:24:1) and precipitated by adding polyacryl carrier (Molecular Research Center, Inc., Cincinnati, Ohio) and ethanol. Genomic (non-fragmented) DNA was extracted from the pellet with DNAzol (Molecular Research Center, Inc.) and the polyacryl carrier. DNA content was measured using PicoGreen (Molecular Probes, Inc., Eugene, Oreg.) on a SpectraMax Gemini Spectrofluorometer (Molecular Devices, Sunnyvale, Calif.). Normalized by fat depot weight, both fragmented and genomic DNA from each sample were loaded on 1.5% agarose gel (pre-stained with 1:10,000 SYBR Green, Molecular Probes, Eugene, Oreg.) for electrophoresis. Apoptosis was verified by the presence of a DNA ladder pattern, which was visualized by a Fluor Chem 8000 fluorescence imaging system (Alpha Innotech, San Leandro, Calif.).

Statistical Analysis: The study was analyzed as a 2×2×2 factorial design (N=5/cell). Analysis of variance (SAS) was used to determine significance of main effects of gender, genotype (WT vs. GDF-8 KO) and clenbuterol (0 vs. 200 ppm) and interactions. Statistically significant differences are defined at the 95% confidence interval.

Results

Food Intake, Body Weight, Weight Gain and Feed Efficiency: There were no significant differences in food intake between genotypes or treatments, but there was a significant difference between females and males in food intake, with females eating less than males (p<0.001; Table 3). There was a significant difference in final body weight (FBW) (P<0.01), but not in weight gain, between females and males (Table 3). There was a difference in both FBW (p<0.05) and weight gain (p<0.05) between WT and GDF-8 KO genotypes.

TABLE 3
Final body weight, daily weight gain and daily food intake
in female and male WT and GDF-8 KO mice fed 0 or 200 ppm
clenbuterol for 21 days (Mean ± SEM).

| | | | | Feed |
|---|---|---|---|---|
| | | | | Efficiency |
| | Final Body | | Food Intake | (g feed/ |
| | Wt (g) | Wt Gain (g/d) | (g/d) | g wt gain) |
| Female | 26.0 ± 0.5** | 0.14 ± 0.01 | 4.48 ± 0.085** | 40.4 ± 4.1 |
| Male | 33.3 ± 0.8** | 0.15 ± 0.015 | 5.14 ± 0.14** | 40.3 ± 5.8 |
| WT | 28.6 ± 0.9* | 0.16 ± 0.015* | 4.89 ± 0.12 | 36.2 ± 3.8 |
| GDF-8 KO | 30.6 ± 1.2* | 0.13 ± 0.01* | 4.74 ± 0.15 | 44.4 ± 5.8 |
| Control | 29.4 ± 0.9 | 0.03 ± 0.01* | 4.83 ± 0.12 | 46.3 ± 5.2 |
| Clenbuterol | 29.9 ± 1.2 | 0.165 ± 0.01* | 4.78 ± 0.16 | 34.4 ± |
| | | | | 4.4† |

*p < 0.05;
**p < 0.01;
†p = 0.07

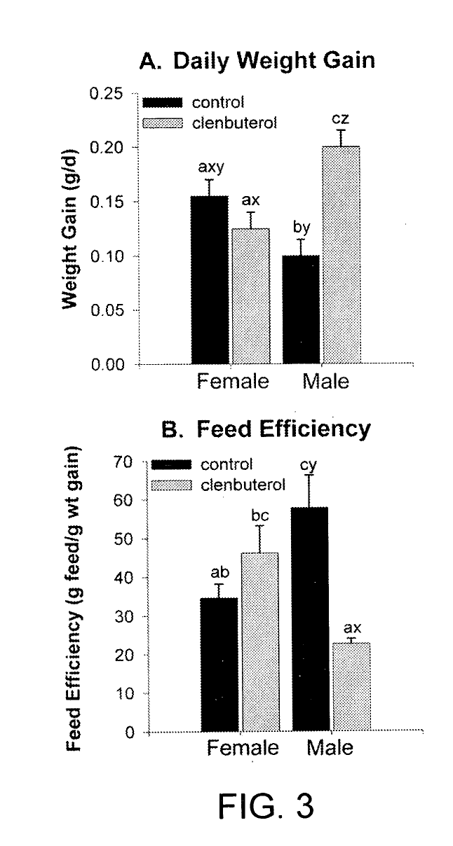

Compared to control, clenbuterol treatment increased weight gain (p<0.05), but did not significantly affect FBW (Table 3). There was a significant gender x treatment interaction for weight gain, showing that clenbuterol significantly increased weight gain in males, but not in females (FIG. 3).

Feed efficiency was calculated as the total food intake (g)/cumulative weight gain (g). There was a trend (p=0.07) for clenbuterol to improve feed efficiency (g feed/g weight gain) (Table 3). There was also a significant gender x treatment interaction for feed efficiency (p<0.001), showing that clenbuterol significantly improved feed efficiency in males (p=0.002), but not in females (FIG. 3).

Body Composition and Tissue Weights: Analysis of main effects for body composition (Table 4) showed that clenbuterol had no effect on BMD, BMC, area, lean weight, fat weight, or fat %. Compared to WT mice, GDF-8 KO mice had higher BMD (p<0.05), BMC (p<0.001), area (p<0.001) and lean weight (p<0.01) and lower fat weight and fat % (p<0.01). Compared to females, males had higher BMD (p<0.001), BMC (p<0.001), area (p<0.01) and lean weight (p<0.001) and had lower fat % (p<0.05). There were no significant interactions.

TABLE 4
Body Composition Analysis of female and male WT and GDF-8 KO mice fed 0
or 200 ppm clenbuterol for 21 days by PIXImus Densitometry (Mean ± SEM).

| | Control | Clenbuterol | WT | GDF-8 KO | Female | Male |
|---|---|---|---|---|---|---|
| Bone Mineral | 0.06 ± 0.001 | 0.06 ± 0.001 | 0.056 ± 0.01 | 0.06 ± 0.01 | 0.05 ± .001 | 0.06 ± .001*** |
| Density (g) | | | | | | |
| Bone Mineral | 0.53 ± 0.02 | 0.52 ± 0.02 | 0.48 ± 0.02 | 0.56 ± 0.02** | 0.47 ± 0.01 | 0.58 ± 0.01*** |
| Content (g) | | | | | | |
| Area (cm²) | 9.0 ± 0.2 | 9.2 ± 0.2 | 8.6 ± 0.2 | 9.6 ± 0.2** | 8.6 ± 0.2 | 9.6 ± 0.2** |
| Lean (g) | 22.2 ± 0.9 | 22.7 ± 1.1 | 21.0 ± 0.8 | 23.9 ± 1.1** | 19.1 ± 0.6 | 25.7 ± 0.6*** |
| Fat (g) | 4.5 ± 0.3 | 4.1 ± 0.2 | 4.9 ± 0.3 | 3.7 ± 0.2** | 4.2 ± 0.3 | 4.4 ± 0.2 |
| % Fat | 17.0 ± 1.2 | 15.7 ± 1.0 | 19.2 ± 1.1 | 13.5 ± 0.7*** | 18.0 ± 1.3 | 14.7 ± 0.8* |

Main effect differences:
*p < 0.05;
**p < 0.01;
***p < .001

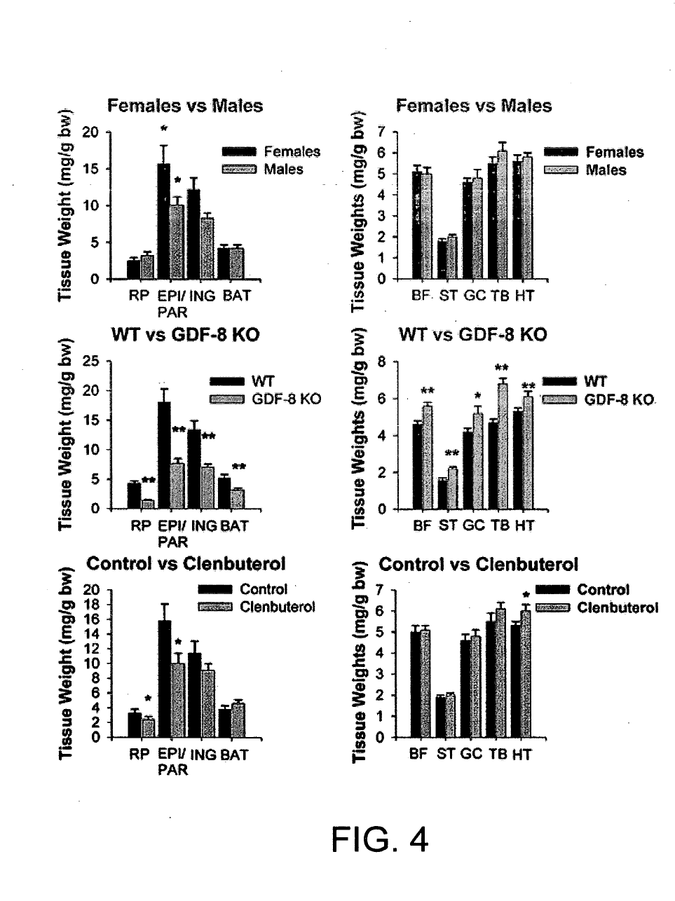

FIG. 4 shows the main effects for adipose and muscle tissue weights expressed per unit body weight. The PAR fat pad in females was larger than the EPI fat pad in males (p<0.05), but there were no differences between males and females in muscle weights when corrected for body weight. In GDF-8 KO mice both the actual weights (not shown) and weights per g body weight of all three white adipose tissues and BAT were lower than in WT mice (p<0.01 and p<0.05 respectively). The actual weights (not shown) and weight per g body weight of all dissected muscles and of the heart were higher in GDF-8 KO mice (p<0.01). Clenbuterol did not affect the actual weights or weight per g body weight of any of the dissected muscles, but mice treated with clenbuterol had smaller RPI and EPI/PAR (p<0.05) fat pads when corrected for body weight. Heart weight was significantly increased in clenbuterol treated mice (p<0.01). There were no significant interactions.

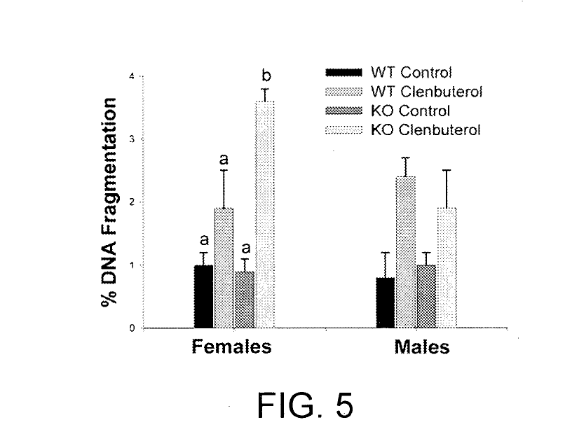

Adipose Tissue Apoptosis: Adipose tissue apoptosis (percent DNA fragmentation) was measured in the EPI/PAR and RP white fat pads. In both tissues clenbuterol treatment increased apoptosis compared to control (p<0.01, Table 5). There were no genotype or gender effects on apoptosis, but there was a significant gender x genotype x treatment interaction. The gender x genotype x treatment interaction resulted in a clenbuterol-induced increase in apoptosis that was significant only in females, however, the clenbuterol-treated males had similar increases in apoptosis (FIG. 5).

TABLE 5
EPI/PAR and RP fat pad apoptosis in female and male WT and GDF-8 KO mice
fed 0 or 200 ppm clenbuterol for 21 days (Mean ± SEM).

| | | | | % | | | % | |
|---|---|---|---|---|---|---|---|---|
| | % DNA Fragmentation | | | DNA Fragmentation | | | DNA Fragmentation | |
| Treatment | EPI/PAR | RP | Genotype | EPI/PAR | RP | Sex | EPI/PAR | RP |
| Control | 0.9 ± 0.1** | 0.9 ± 0.2** | WT | 1.5 ± 0.2 | 1.6 ± 0.3 | F | 1.8 ± 0.3 | 1.4 ± 0.3 |
| Clenbuterol | 2.5 ± 0.2** | 2.5 ± 0.2** | GDF-8 KO | 1.8 ± 0.3 | 1.8 ± 0.4 | M | 1.5 ± 0.2 | 2.0 ± 0.4 |

**Column means are different, p < 0.01

Serum Leptin Concentrations: Serum leptin concentrations were lower in the GDF-8 KO mice compared to WT mice (p<0.03); however, clenbuterol did not significantly affect leptin concentrations (Table 6). Leptin concentrations were also lower in the males compared to females (p<0.02).

TABLE 6
Serum Leptin Concentration (ng/ml) in female and male WT and GDF-8 KO
mice fed 0 or 200 ppm clenbuterol for 20 days (Mean ± SEM).

| Treatment | Leptin (ng/ml) | Genotype | Leptin (ng/ml) | Sex | Leptin (ng/ml) |
|---|---|---|---|---|---|
| Control | 5.31 ± 1.10 | WT | 5.97 ± 0.85* | F | 5.94 ± 1.06* |
| Clenbuterol | 3.98 ± 0.49 | GDF-8 KO | 3.50 ± 0.73* | M | 3.27 ± 0.28* |

*Column means are different, p < 0.05

Discussion:

Clenbuterol did not affect food intake, body weight or body composition in either WT or GDF-8 KO mice, although it did increase cumulative weight gain and improve feed efficiency in a gender-specific manner. Male mice fed the clenbuterol-containing diet gained twice as much weight and were more than 250% more efficient compared to males fed control diet, whereas there were no differences in female mice. Clenbuterol also had no effect on weights of specific skeletal muscles, but it decreased weight of the EPI/PAR fat pad and increased heart weight. In a previous study, however, it was found that neither clenbuterol or ractopamine had no effect on muscle weight or on body protein or fat content in ICR mice (Page, K., M. A. Della-Fera, C. L. Li, M. J. Azain, T. D. Pringle, D. Hartzell, and C. A. Baile, Effects of Clenbuterol on Muscle, Adipose Tissue and Adipocyte Apoptosis in Myostatin Knockout Mice, J Anim Sci, 2004, in press.). Since the same lack of effect has been shown in a different strain of mice, it is less likely that previous findings were simply due to strain-related differences.

As in our previous study, clenbuterol increased apoptosis in both the EPI/PAR and RP fat pads. Although there appeared to be a difference in sensitivity between males and females, this was likely due to the higher variability in samples from males compared to females, since there were increases in apoptosis in clenbuterol-treated males that were similar to those in females.

The differences between GDF-8 KO and WT mice in body weight, weight gain, body composition and tissue weights are typical of animals with GDF-8 gene knockout or mutation (McPherron, A. C., A. M. Lawler, and S. J. Lee, Regulation of skeletal muscle mass in mice by a new TGF-beta superfamily member, Nature, 1997. 387(6628): p. 83-90; McPherron, A. C. and S. J. Lee, Suppression of body fat accumulation in myostatin-deficient mice, J Clin Invest, 2002. 109(5): p. 595-601). Serum leptin concentrations were not affected by clenbuterol treatment; however, GDF-8 KO mice had lower leptin levels compared to WT mice. This corresponds to a previous study in which serum leptin concentrations were lower in GDF-8 KO mice, a finding that is likely due to the reduced amount of adipose tissue mass in GDF-8 KO mice (J Clin Invest, 2002. 109(5): p. 595-601).

The increased muscle mass in GDF-8 KO mice is due to both hypertrophy and hyperplasia (Nature, 1997. 387(6628): p. 83-90). Although it is conceivable that adult GDF-8 KO mice have reached their maximum physiological potential for increased muscle mass, identifying a molecular mechanism that defines that potential could have important implications for livestock production.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

Therefore, having thus described the invention, at least the following is claimed:

1. A method for increasing the rate of apoptosis in adipose tissue cells in a host, comprising the step of administering an effective amount of at least one catecholamine to the host.

2. The method of claim 1, wherein the adipose tissue is white adipose tissue.

3. The method of claim 1, wherein the catecholamine is selected from compounds that bind to a beta-1-adrenergic receptor, beta-2-adrenergic receptor agonist, both beta-1 and beta-2-adrenergic receptors, a beta-3-adrenergic receptor, and combinations thereof.

4. The method of claim 3, wherein the catecholamine is selected from dobutamine and benzamide, 2-hydroxy-5-[2-[[2-hydroxy-3-[4-[1-methyl-4-(trifluoromethyl)-1H-imidazol-2-yl]phenoxy]propyl]amino]ethoxy]-, monomethanesulfonate (salt) (9CI).

5. The method of claim 3, wherein the catecholamine is selected from clenbuterol; cimaterol; 2-butanol, 1-[(2,3-dihydro-7-methyl-1H-inden-4-yl)oxy]-3-[(1-methylethyl)amino]-, (2R,3S)-rel-(9CI); procaterol; terbutaline; formoterol; salmeterol; ractopamine; and salbutamol.

6. The method of claim 3, wherein the catecholamine is selected from 1,3-benzodioxole-2,2-dicarboxylic acid, 5-[(2R)-2-[[(2R)-2-(3-chlorophenyl)-2-hydroxyethyl]amino]propyl]-, disodium salt (9CI)); acetic acid, [4-[(2R)-2-[[(2R)-2-(3-chlorophenyl)-2-hydroxyethyl]amino]propyl]phenoxy]-, rel-(9CI); 2H-benzimidazol-2-one, 4-[3-[(1,1-dimethylethyl)amino]-2-hydroxypropoxy]-1,3-dihydro-(9CI); and benzoic acid, 4-[(2R)-2-[[(2R)-2-hydroxy-2-phenylethyl]amino]propyl]-, methyl ester, rel-, (2E)-2-butenedioate (2:1) (salt) (9CI).

7. The method of claim 1, wherein the host is a human.

8. The method of claim 1, wherein the host is selected from dogs, cats, chickens, pigs, hogs, and cows.

9. A pharmaceutical composition comprising of at least one catecholamine in combination with a pharmaceutically acceptable carrier, wherein the at least one catecholamine is present in a dosage level effective to increase the rate of apoptosis in adipose tissue cells in a host.

10. The pharmaceutical composition of claim 8, wherein the adipose tissue is white adipose tissue.

11. The pharmaceutical composition of claim 10, wherein the host is selected from cats, dogs, chickens, pigs, hogs, and cows.

12. The pharmaceutical composition of claim 8, wherein the catecholamine is selected from dobutamine and benzamide, 2-hydroxy-5-[2-[[2-hydroxy-3-[4-[1-methyl-4-(trifluoromethyl)-1H-imidazol-2-yl]phenoxy]propyl]amino]ethoxy]-, monomethanesulfonate (salt) (9CI).

13. The pharmaceutical composition of claim 8, wherein the catecholamine is selected from clenbuterol; cimaterol; 2-butanol, 1-[(2,3-dihydro-7-methyl-1H-inden-4-yl)oxy]-3-[(1-methylethyl)amino]-, (2R,3S)-rel-(9CI); procaterol, terbutaline, formoterol; salmeterol; ractopamine; and salbutamol.

14. The pharmaceutical composition of claim 8, wherein the catecholamine is selected from 1,3-benzodioxole-2,2-dicarboxylic acid, 5-[(2R)-2-[[(2R)-2-(3-chlorophenyl)-2-hydroxyethyl]amino]propyl]-, disodium salt (9CI)); acetic acid, [4-[(2R)-2-[[(2R)-2-(3-chlorophenyl)-2-hydroxyethyl]amino]propyl]phenoxy]-, rel-(9CI); 2H-benzimidazol-2-one, 4-[3-[(1,1-dimethylethyl)amino]-2-hydroxypropoxy]-1,3-dihydro-(9CI); and benzoic acid, 4-[(2R)-2-[[(2R)-2-hydroxy-2-phenylethyl]amino]propyl]-, methyl ester, rel-, (2E)-2-butenedioate (2:1) (salt) (9CI).

15. A method of reducing adipose tissue mass in a host comprising administering to the host an effective amount of at least one catecholamine.

16. The method of claim 15, wherein the adipose tissue is white adipose tissue.

17. The method of claim 16, wherein the catecholamine is selected from compounds that bind to a beta-1-adrenergic receptor, beta-2-adrenergic receptor agonist, both beta-1 and beta-2-adrenergic receptors, a beta-3-adrenergic receptor, and combinations thereof.

18. The method of claim 15, wherein the catecholamine is selected from dobutamine and benzamide, 2-hydroxy-5-[2-[[2-hydroxy-3-[4-[1-methyl-4-(trifluoromethyl)-1H-imidazol-2-yl]phenoxy]propyl]amino]ethoxy]-, monomethanesulfonate (salt) (9CI).

19. The method of claim 15, wherein the catecholamine is selected from clenbuterol;
cimaterol; 2-butanol, 1-[(2,3-dihydro-7-methyl-1H-inden-4-yl)oxy]-3-[(1-methylethyl)amino]-, (2R,3S)-rel-(9CI); procaterol; terbutaline; formoterol, salmeterol; ractopamine; and salbutamol.

20. The method of claim 15, wherein the catecholamine is selected from 1,3-benzodioxole-2,2-dicarboxylic acid, 5-[(2R)-2-[[(2R)-2-(3-chlorophenyl)-2-hydroxyethyl]amino]propyl]-, disodium salt (9CI)); acetic acid, [4-[(2R)-2-[[(2R)-2-(3-chlorophenyl)-2-hydroxyethyl]amino]propyl]phenoxy]-, rel-(9CI); 2H-benzimidazol-2-one, 4-[3-[(1,1-dimethylethyl)amino]-2-hydroxypropoxy]-1,3-dihydro-(9CI); and benzoic acid, 4-[(2R)-2-[[(2R)-2-hydroxy-2-phenylethyl]amino]propyl]-, methyl ester, rel-, (2E)-2-butenedioate (2:1) (salt) (9CI).

21. The method of claim 15, wherein the catecholamine is selected from clenbuterol and ractopamine.

22. A pharmaceutical composition comprising of at least one catecholamine in combination with a pharmaceutically acceptable carrier, wherein the at least one catecholamine is present in a dosage level effective to reduce adipose tissue mass in a host.

23. The pharmaceutical composition of claim 22, wherein the adipose tissue is white adipose tissue.

24. The pharmaceutical composition of claim 23, wherein the catecholamine is selected from dobutamine; benzamide; 2-hydroxy-5-[2-[[2-hydroxy-3-[4-[1-methyl-4-(trifluoromethyl)-1H-imidazol-2-yl]phenoxy]propyl]amino]ethoxy]-, monomethanesulfonate (salt) (9CI); ractopamine; clenbuterol; cimaterol, 2-butanol, 1-[(2,3-dihydro-7-methyl-1H-inden-4-yl)oxy]-3-[(1-methylethyl)amino]-, (2R,3S)-rel-(9CI); procaterol; terbutaline; formoterol; salmeterol; salbutamol; 1,3-benzodioxole-2,2-dicarboxylic acid, 5-[(2R)-2-[[(2R)-2-(3-chlorophenyl)-2-hydroxyethyl]amino]propyl]-, disodium salt (9CI)); acetic acid, [4-[(2R)-2-[[(2R)-2-(3-chlorophenyl)-2-hydroxyethyl]amino]propyl]phenoxy]-, rel-(9CI), 2H-benzimidazol-2-one, 4-[3-[(1,1-dimethylethyl)amino]-2-hydroxypropoxy]-1,3-dihydro-(9CI); benzoic acid, 4-[(2R)-2-[[(2R)-2-hydroxy-2-phenylethyl]amino]propyl]-, methyl ester, rel-, (2E)-2-butenedioate (2:1) (salt) (9CI) and combinations thereof.

25. The pharmaceutical composition of claim 22, wherein the catecholamine is selected from clenbuterol and ractopamine.
